(19) United States
(12) Patent Application Publication
(10) Pub. No.: US 2024/0009255 A1
(43) Pub. Date: Jan. 11, 2024

(54) COMPOSITION FOR PREVENTING, TREATING OR AMELIORATING EOSINOPHILIC INFLAMMATORY DISEASES OR TH2 HYPERSENSITIVITY IMMUNE DISEASES COMPRISING LACTOCOCCUS LACTIS-DERIVED VESICLES

(71) Applicant: MD HEALTHCARE INC., Seoul (KR)

(72) Inventor: Yoon-Keun KIM, Paju-Si, Gyeonggi-do (KR)

(73) Assignee: MD HEALTHCARE INC., Seoul (KR)

(21) Appl. No.: 18/254,931

(22) PCT Filed: Oct. 19, 2021
(86) PCT No.: PCT/KR2021/014540

§ 371 Date: May 30, 2023

(30) Foreign Application Priority Data
Nov. 30, 2020 (KR) 10-2020-0164938
Oct. 18, 2021 (KR) 10-2021-0138471

(51) Int. Cl. A61K 35/744; A61P 37/08

(52) U.S. Cl. CPC A61K 35/744; A61P 37/08

(57) ABSTRACT

Provided is a composition for preventing, treating or alleviating eosinophilic inflammatory diseases and Th2 hypersensitivity immune diseases, comprising Lactococcus lactis-derived vesicles, and it was confirmed that, when Lactococcus lactis-derived vesicles according to the presently claimed subject matter were administered to a Th2 hypersensitivity immune disease mouse model, the vesicles inhibited eosinophilic inflammation and the consequent functional and histopathological changes. It was also confirmed that the vesicles inhibited the secretion of Th2 cytokines such as IL-5 and IL-13 in T cells of the Th2 hypersensitivity immune disease mouse model, which is shown by inducing IL-12 secretion that suppresses a Th2 immune response in dendritic cells, which are antigen-presenting cells. Thus, the Lactococcus lactis-derived vesicles according to the presently claimed subject matter are expected to be effectively used in a composition and the like for preventing, treating or alleviating eosinophilic inflammatory diseases and Th2 hypersensitivity immune diseases.

### TECHNICAL FIELD

The present invention relates to a composition for preventing, treating, or alleviating eosinophilic inflammatory diseases or Th2 hypersensitivity immune diseases, comprising Lactococcus lactis-derived vesicles.

This application claims priority to and the benefit of Korean Patent Application No. 10-2020-0164938, filed on Nov. 30, 2020, and Korean Patent Application No. 10-2021-0138471, filed on Oct. 18, 2021, the disclosures of which are incorporated herein by reference in their entirety.

### BACKGROUND ART

Eosinophilic inflammatory diseases are the group of diseases that are presumed to find a large number of eosinophils in a lesion, or that the eosinophils would play a critical pathophysiological role in the onset of a disease. Eosinophilic inflammatory diseases may occur by various causes, and the type of disease may vary depending on the site of its origin. For example, compared to eosinophilic pneumonia, which is a representative eosinophilic inflammatory disease, finding the infiltration of numerous eosinophils in the lung parenchyma, allergic bronchopulmonary aspergillosis is localized in the airway.

Eosinophilic pneumonia of the eosinophilic inflammatory diseases may be caused by identified causes such as parasite infection or medicines, but some types, for example, Leffler syndrome or chronic eosinophilic pneumonia, and Churg-Strauss allergic granulomatosis, may be caused by unidentified causes. When inflammation occurs, inflammatory cells such as monocytes, giant cells, and eosinophils infiltrate alveoli, sometimes accompanied by interstitial infiltrates in the lung. In addition, allergic asthma is a chronic inflammatory disorder of the airway associated with a type 2 helper T (Th2) cell-dependent immune response, eosinophilia, and IgE production, and in the onset of allergic asthma, Th2 cells produce various types of cytokines including interleukin (IL)-5, IL-9 and IL-13, modulating an immune response. Among these factors, IL-5 and IL-9 contribute to eosinophilia and mast cell proliferation, whereas IL-13 is involved in mucus hypersecretion, and dendritic cells play an important role in antigen presentation and T cell differentiation in a lymphoid organ as a result of allergen exposure.

Generally, dendritic cells produce not only IL-4, which is a cytokine inducing differentiation into Th2 cells, but also IL-12 inducing the differentiation into Th1 cells, thereby regulating Th1/Th2 balance. Among various factors, bacteria are also known to interact with dendritic cells to regulate allergic airway inflammation.

Changes in abundance and diversity of commensal bacteria may affect asthma exacerbation by contributing to inflammation and remodeling of the lungs. Particularly, probiotics (defined as live microorganisms that have a beneficial effect on the host) have been proposed to prevent an allergic reaction by several mechanisms. The probiotics are mainly associated with the development of regulatory T cells that produce IL-10 known as an immunosuppressive cytokine. As, among the probiotics, Bifidobacterium breve attenuates airway inflammation by inducing IL-10-producing T cells, such non-pathogenic and non-invasive bacteria are already used in biotechnological applications, showing that probiotics have an advantage as well as long-term safety.

Meanwhile, it is known that there are 100 trillion microorganisms coexisting in the human body, which is 10 times higher than human cells, and the number of microbial genes is known to be over 100 times the number of human genes. Microbiota (or microbiome) refers to a microbial community including bacteria, archaea, and eukaryotes, which are present in given habitats, intestinal microbiota plays a critical role in human physiology, and are known to have a great influence on human health and diseases through interaction with human cells. Bacteria coexisting in our body secretes nanometer-sized vesicles in order to exchange information on genes, proteins, and the like with other cells. The mucosa forms a physical defense membrane through which particles having a size of 200 nanometers (nm) or more cannot pass, so that bacteria coexisting in the mucosa cannot pass through the mucosa, but vesicles derived from bacteria have a size of 200 nanometers or less and are absorbed into our bodies after relatively freely passing through the mucosa. Although bacteria-derived vesicles are secreted from bacteria, they differ from bacteria in terms of their constituents, absorption rate in the body, and risk of side effects, and therefore, the use of bacteria-derived vesicles is completely different from that of living cells or has a significant effect.

Extracellular vesicles (EVs; membrane-bound organelles that deliver cargos of proteins, nucleic acids, lipids, and metabolites) are secreted by all cell types including eukaryotes and prokaryotes under physiological and pathological conditions. Since these novel molecules have a function of interacting between cells as well as an effect associated with immune systems, EVs are known to be involved in various human diseases such as cancer, metabolic disorders, and allergic diseases.

Bacteria of the genus Lactococcus are gram-positive cocci that secrete lactic acid, and among these bacteria, Lactococcus lactis is known as a bacterium important for fermentation of dairy products such as cheese, fermented vegetables, and alcoholic beverages, and may be isolated from materials fermented from milk and plants.

However, there has been no known therapeutic effect of vesicles derived from bacteria of the genus Lactococcus on eosinophilic inflammatory diseases or Th2 hypersensitivity immune diseases.

### DISCLOSURE

### Technical Problem

Therefore, the present invention is directed to providing a pharmaceutical composition for preventing or treating eosinophilic inflammatory diseases or Th2 hypersensitivity immune diseases, comprising Lactococcus lactis-derived vesicles as an active ingredient.

The present invention is also directed to providing an inhalant composition for preventing or treating eosinophilic inflammatory diseases or Th2 hypersensitivity immune diseases, comprising Lactococcus lactis-derived vesicles as an active ingredient.

The present invention is also directed to providing a food composition for preventing or alleviating eosinophilic inflammatory diseases or Th2 hypersensitivity immune diseases, comprising Lactococcus lactis-derived vesicles as an active ingredient.

The present invention is also directed to providing a quasi-drug composition for preventing or alleviating eosinophilic inflammatory diseases or Th2 hypersensitivity immune diseases, comprising Lactococcus lactis-derived vesicles as an active ingredient.

The present invention is also directed to providing a composition for delivering a drug for treating respiratory diseases, comprising Lactococcus lactis-derived vesicles as an active ingredient.

However, a technical problem to be achieved by the present invention is not limited to the aforementioned problems, and the other problems that are not mentioned may be clearly understood by a person skilled in the art from the following description.

### Technical Solution

To achieve the object of the present invention as described above, the present invention provides a pharmaceutical composition for preventing or treating eosinophilic inflammatory diseases or Th2 hypersensitivity immune diseases, comprising Lactococcus lactis-derived vesicles as an active ingredient.

In addition, the present invention provides an inhalant composition for preventing or treating eosinophilic inflammatory diseases or Th2 hypersensitivity immune diseases, comprising Lactococcus lactis-derived vesicles as an active ingredient.

In addition, the present invention provides a food composition for preventing or alleviating eosinophilic inflammatory diseases or Th2 hypersensitivity immune diseases, comprising Lactococcus lactis-derived vesicles as an active ingredient.

In addition, the present invention provides a quasi-drug composition for preventing or alleviating eosinophilic inflammatory diseases or Th2 hypersensitivity immune diseases, comprising Lactococcus lactis-derived vesicles as an active ingredient.

In one embodiment of the present invention, the eosinophilic inflammatory diseases are allergic inflammatory diseases, and the allergic inflammatory diseases may comprise eosinophilic drug allergy, eosinophilic asthma, allergic rhinitis, and atopic dermatitis, but the present invention is not limited thereto.

In another embodiment of the present invention, the eosinophilic inflammatory diseases are eosinophilic inflammatory diseases of unknown etiology, and the eosinophilic inflammatory diseases of unknown etiology may comprise eosinophilic cardiomyopathy, eosinophilic colitis, eosinophilic enteritis, eosinophilic esophagitis, eosinophilic gastritis, eosinophilic pneumonia, eosinophilic bronchitis, eosinophilic fasciitis, hypereosinophilic syndrome, and Churg-Strauss syndrome or eosinophilic granulomatosis with polyangiitis, but the present invention is not limited thereto.

In still another embodiment of the present invention, the Th2 hypersensitivity immune diseases may comprise atopic dermatitis, allergic conjunctivitis, allergic rhinitis, allergic asthma, hypersensitivity pneumonitis, food allergy, drug allergy, and anaphylaxis, but the present invention is not limited thereto.

In yet another embodiment of the present invention, the eosinophilic inflammatory diseases may be respiratory diseases showing type 2 helper T (Th2) cell hypersensitivity, but the present invention is not limited thereto.

In yet another embodiment of the present invention, the respiratory diseases showing Th2 cell hypersensitivity may comprise atopic asthma, allergic rhinitis, eosinophilic bronchitis, and hypersensitivity pneumonitis, but the present invention is not limited thereto.

In yet another embodiment of the present invention, the eosinophilic inflammatory diseases or Th2 hypersensitivity immune diseases may be respiratory diseases characterized by excessive secretion of mucus to the respiratory organs, but the present invention is not limited thereto.

In yet another embodiment of the present invention, the respiratory diseases characterized by excessive secretion of mucus to the respiratory organs may comprise chronic rhinitis, chronic sinusitis, and chronic bronchitis, but the present invention is not limited thereto.

In yet another embodiment of the present invention, the Lactococcus lactis-derived vesicles may inhibit airway hyperresponsiveness, but the present invention is not limited thereto.

In yet another embodiment of the present invention, the vesicles may have an average diameter of 10 to 200 nm, but the present invention is not limited thereto.

In yet another embodiment of the present invention, the vesicles may be secreted naturally or artificially from Lactococcus lactis, but the present invention is not limited thereto.

In addition, the present invention provides a composition for delivering a drug for treating respiratory diseases, comprising Lactococcus lactis-derived vesicles as an active ingredient.

In addition, the present invention provides a method of preventing or treating eosinophilic inflammatory diseases or Th2 hypersensitivity immune diseases, comprising administering a composition comprising Lactococcus lactis-derived vesicles as an active ingredient to a subject.

In addition, the present invention provides a use of a composition comprising Lactococcus lactis-derived vesicles as an active ingredient for preventing or treating eosinophilic inflammatory diseases or Th2 hypersensitivity immune diseases.

In addition, the present invention provides a use of Lactococcus lactis-derived vesicles for preparing a medicine for preventing or treating eosinophilic inflammatory diseases or Th2 hypersensitivity immune diseases.

In addition, the present invention provides a method of delivering a drug for treating respiratory diseases, the method comprising administering a composition comprising Lactococcus lactis-derived vesicles containing a drug for treating respiratory diseases to be targeted as an active ingredient to a subject.

In addition, the present invention provides a use of a composition comprising Lactococcus lactis-derived vesicles as an active ingredient for delivering a drug for treating respiratory diseases.

In addition, the present invention provides a use of Lactococcus lactis-derived vesicles for preparing a medicine for delivering a drug for treating respiratory diseases.

### Advantageous Effects

When Lactococcus lactis-derived vesicles according to the present invention are administered to a mouse model with a Th2 hypersensitivity immune disease, IFN-γ secretion increased in Th1 cells, IL-5 and IL-13 secretion decreased in Th2 cells, and IL-12p70 secretion increased in dendritic cells, confirming that a shift of the immune response from a Th2 cell immune response to a Th1 cell immune response is induced. In addition, the Lactococcus lactis-derived vesicles according to the present invention are effective in inhibiting airway hypersensitivity, reducing eosinophilic infiltration to lung tissue, and inhibiting mucus production in the lungs, so it is expected that the Lactococcus lactis-derived vesicles according to the present invention can be effectively used for a composition for preventing, treating, or alleviating eosinophilic inflammatory diseases or Th2 hypersensitivity immune diseases.

### DESCRIPTION OF DRAWINGS

FIGS. 1A and 1B are views that confirm the characteristics of Lactococcus lactis-derived vesicles according to an embodiment of the present invention, wherein

FIG. 1A is an image of vesicles using a transmission electron microscope (scale bar: nm), and FIG. 1B is a view that confirms a protein component in the vesicles.

FIGS. 2A and 2B are views that confirm the characteristics of Bifidobacterium breve-derived vesicles according to one embodiment of the present invention, wherein FIG. 2A is an image of vesicles using a transmission electron microscope (scale bar: nm), and FIG. 2B is a view that confirms a protein component in the vesicles.

FIG. 3 is a view illustrating an experimental protocol for assessing therapeutic effects of Lactococcus lactis-derived vesicles and Bifidobacterium breve-derived vesicles in mouse models with Th2 hypersensitivity immune diseases according to one embodiment of the present invention.

FIGS. 4A and 4B are views that confirm the effects of Lactococcus lactis (L. lactis)-derived vesicles and Bifidobacterium breve (B. breve)-derived vesicles on eosinophilic infiltration in mouse models with Th2 hypersensitivity immune diseases according to one embodiment of the present invention, wherein FIG. 4A is an image of cells stained with H&E in bronchoalveolar lavage fluid (BALF), and FIG. 4B is a view illustrating the number of eosinophils in BALF (n=5, **P<0.01, ***P<0.001, and n.s. means not significant).

FIG. 5 is a view that confirms the effects of L. lactis-derived vesicles and B. breve-derived vesicles on lung dysfunction (airway hypersensitivity) caused by eosinophilic inflammation in mouse models with Th2 hypersensitivity immune diseases according to one embodiment of the present invention (n=5, *P<0.05, **P<0.01).

FIG. 6 is a view that evaluates the effects of L. lactis-derived vesicles and B. breve-derived vesicles on a histological change in the lungs in mouse models with Th2 hypersensitivity immune diseases according to one embodiment of the present invention, in which FIG. 6A is a view that confirms a change in mucus production of the lungs (scale bar: 50 pin), and FIG. 6B is a view of the quantifying the result of FIG. 6A (*P<0.05, **P<0.01, ***P<0.001, and n.s. means not significant).

FIGS. 7A and 7B are views that assess Th1 and Th2 immune response regulatory effects of L. lactis-derived vesicles and B. breve-derived vesicles in mouse models with Th2 hypersensitivity immune diseases according to one embodiment of the present invention, wherein FIG. 7A shows the result of measuring IFN-γ, which is a Th1 cytokine, in BALF, and FIG. 7B is a view obtained by measuring IL-5 and IL-13, which are Th2 cytokines inducing eosinophilic infiltration (n=5, *P<0.05, **P<0.01, ***P<0.001, and n.s. means not significant).

FIGS. 8A and 8B are views that assess Th1 and Th2 immune response-regulatory effects of L. lactis-derived vesicles and B. breve-derived vesicles in mouse models with Th2 hypersensitivity immune diseases according to one embodiment of the present invention, wherein FIG. 8A is a result obtained by measuring the secretion of IFN-γ, which is a Th1 cytokine, in T cells isolated from lung tissue, and FIG. 8B is a view obtained by measuring the secretion of IL-5 and IL-13, which are Th2 cytokines, in T cells isolated from lung tissue (n=5, *P<0.05, **P<0.01, ***P<0.001, and n.s. means not significant).

FIGS. 9A to 9C are views that illustrate the evaluation of Th1 and Th2 immune response-regulatory mechanisms by L. lactis-derived vesicles and B. breve-derived vesicles according to one embodiment of the present invention, wherein FIG. 9A shows an experimental protocol for evaluating a cytokine secretion pattern in T cells in peripheral blood isolated from a healthy control, FIG. 9B shows a result obtained by measuring the secretion of IFN-γ, which is a Th1 cytokine, secreted from T cells in peripheral blood by stimulation of anti-CD3/28 antibody, and FIG. 9C is a view that confirms secretion levels of IL-4 and IL-5, which are Th2 cytokines secreted from T cells in peripheral blood by stimulation of anti-CD3/28 antibody (n=6, ***P<0.001, and n. s. means not significant).

FIGS. 10A and 10B are views that illustrate the evaluation of Th1 and Th2 immune response-regulatory mechanisms by L. lactis-derived vesicles and B. breve-derived vesicles according to one embodiment of the present invention, wherein FIG. 10A shows an experimental protocol for evaluating a cytokine secretion pattern involved in T cell differentiation in dendritic cells in peripheral blood isolated from a healthy control, and FIG. 10B is a view that confirms a level of IL-12p70 inducing a Th1 immune response (n=6, *P<0.05, ***P<0.001, and n.s. means not significant).

### BEST MODE

In one experimental example of the present invention, it was confirmed that a Lactococcus lactis (L. lactis)-derived vesicle has a membrane consisting of a bilayer, and while a Bifidobacterium breve (B. breve)-derived vesicle shows a single protein band, the L. lactis-derived vesicle shows several protein bands (see Experimental Example 1).

In another experimental example of the present invention, while eosinophilic infiltration to lung tissue and airway hypersensitivity were not inhibited by treatment with B. breve-derived vesicles in animal models with Th2 hypersensitivity immune diseases, eosinophilic infiltration to lung tissue and airway hypersensitivity were inhibited by treatment with L. lactis-derived vesicles. While the treatment with B. breve-derived vesicles had no effect on mucus production in a Th2 hypersensitivity immune disease animal model, it was confirmed that mucus production is significantly reduced by treatment with L. lactis-derived vesicles (see Experimental Example 2).

In still another experimental example of the present invention, the secretion of Th2 cytokines such as IL-5 and IL-13 inducing eosinophilic inflammation is inhibited and the secretion of IFN-γ, which is a Th1 cytokine, is induced by treatment with L. lactis-derived vesicles in Th2 hypersensitivity immune disease animal models, and such an immunoregulatory effect was not observed by treatment with B. breve-derived vesicles. That is, it can be seen that L. lactis-derived vesicles induce immunological homeostasis by inducing a Th2-to-Th1 immune response in a situation where a Th2 immune response is dominant (see Experimental Example 3).

In yet experimental example of the present invention, it can be seen that the secretion of a cytokine such as IL-12p70 inducing a Th1 immune response is significantly increased by L. lactis-derived vesicles acting on antigen-presenting cells, that is, dendritic cells, rather than acting directly on T cells to regulate Th1 and Th2 immune responses, thereby having an immunoregulatory mechanism inhibiting Th2 hypersensitivity (see Experimental Example 4).

Thus, the present invention provides a pharmaceutical composition for preventing or treating eosinophilic inflammatory diseases or Th2 hypersensitivity immune diseases, comprising Lactococcus lactis-derived vesicles as an active ingredient.

The “eosinophil” used herein is a type of white blood cell, and a member of the immune system that serves to confront multicellular parasites and specific infection in mammals. This is involved in the pathogenesis of eosinophilic inflammatory diseases by cytokines secreted by Th2 immune response, such as IL-5, IL-13, etc. The eosinophils are generated in the bone marrow through a hematopoietic process, sent to the blood, then finally differentiate and are no longer proliferated. These are cells having acidophilic granules in the cytoplasm and shown brick-red when stained with the dye, eosin. Small granules in the cytoplasm contain several chemical mediators, such as a peroxidase, an RNAase, a DNAase, a lipase, and a plasminogen. These mediators are secreted by degranulation according to the activation of eosinophils, resulting in histological and functional changes, causing diseases.

The “eosinophilic inflammatory disease” used herein refers to an inflammatory disease occurred by infiltrating eosinophils in tissue by various causes. The eosinophilic inflammatory disease may be an inflammatory disease caused by an allergen, comprising drug allergy, eosinophilic asthma, allergic rhinitis, atopic dermatitis, and the like, but the present invention is not limited thereto.

In addition, the eosinophilic inflammatory diseases may be eosinophilic inflammatory diseases of unknown etiology, comprising eosinophilic cardiomyopathy, eosinophilic colitis, eosinophilic enteritis, eosinophilic esophagitis, eosinophilic gastritis, eosinophilic pneumonia, eosinophilic bronchitis, eosinophilic fasciitis, hypereosinophilic syndrome, Churg-Strauss syndrome or eosinophilic granulomatosis with polyangiitis, and the like, but the present invention is not limited thereto.

The “Th2 hypersensitivity immune diseases” used herein refer to immune diseases mediated by cytokines such as IL-4, IL-5, IL-9, and IL-13, secreted by Th2 cell hypersensitivity caused by a specific antigen. The Th2 hypersensitivity immune diseases caused by Th2 cell hypersensitivity may comprise atopic dermatitis, allergic conjunctivitis, allergic rhinitis, allergic asthma, hypersensitivity pneumonitis, food allergy, drug allergy, anaphylaxis, and the like, but the present invention is not limited thereto.

In the present invention, the eosinophilic inflammatory diseases may be respiratory diseases showing Th2 cell hypersensitivity, and here, the respiratory diseases with Th2 cell hypersensitivity may comprise atopic asthma, allergic rhinitis, eosinophilic bronchitis, hypersensitivity pneumonitis, and the like, but the present invention is not limited thereto.

In the present invention, the eosinophilic inflammatory diseases or Th2 hypersensitivity immune diseases may be respiratory diseases characterized by excessive mucus secretion in respiratory organs, and here, the respiratory diseases characterized by excessive mucus secretion in respiratory organs may comprise chronic rhinitis, chronic sinusitis, and chronic bronchitis, but the present invention is not limited thereto.

“Asthma” used herein is a disease of the “bronchi,” which is the passage connected to the lungs, in which cough, stridor (a wheezing sound when breathing), dyspnea, and chest tightness occur repeatedly because the bronchi become severely narrower due to inflammation of the bronchi when being exposed to a specific causative factor. Asthma also refers to any disease of the lung, characterized by a change in pulmonary airflow associated with airway constriction whatever the cause is (endogenous, exogenous, or both; allergic or non-allergic). The term “asthma” may be used along with one or more adjectives indicating causes.

The term “extracellular vesicle or vesicle” as used herein refers to a structure consisting of a nano-sized membrane secreted from various bacteria. Gram-positive bacteria-derived vesicles such as Lactococcus or Bifidobacterium have peptidoglycan and lipoteichoic acid, which are the components of a bacterial cell wall, and various low-molecular compounds, as well as a protein and a nucleic acid. In the present invention, vesicles are naturally secreted or artificially produced by L. lactis bacteria, and may have an average diameter of, for example, 10 to 200 nm, 10 to 180 nm, 10 to 150 nm, 10 to 120 nm, 10 to 100 nm, 10 to 80 nm, 20 to 200 nm, 20 to 180 nm, 20 to 150 nm, 20 to 120 nm, 20 to 100 nm, or 20 to 80 nm, but the present invention is not limited thereto.

The vesicles may be isolated from a culturing solution comprising bacteria of L. lactis by using one or more methods selected from the group consisting of centrifugation, ultra-high speed centrifugation, high pressure treatment, extrusion, sonication, cell lysis, homogenization, freezing-thawing, electroporation, mechanical decomposition, chemical treatment, filtration by a filter, gel filtration chromatography, free-flow electrophoresis, and capillary electrophoresis. Further, a process such as washing for removing impurities and concentration of obtained vesicles may be further included.

In the present invention, the L. lactis-derived vesicles may inhibit Th2 immune response by increasing the secretion of interferon-γ (IFN-γ) in T cells; or decreasing the secretion of one or more selected from the group consisting of IL-5 and IL-13 in T cells and increasing the secretion of IL-12p70 in dendritic cells, and thus can be used as a composition for preventing, treating, or alleviating eosinophilic inflammatory diseases or Th2 hypersensitivity immune diseases, but the present invention is not limited thereto.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxy cellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As another aspect of the present invention, the present invention provides an inhalant composition for preventing or treating eosinophilic inflammatory diseases or Th2 hypersensitivity immune diseases, comprising Lactococcus lactis-derived vesicles as an active ingredient.

In the inhalant composition of the present invention, the active ingredient may be added to the inhalant as it is, or may be used together with another ingredient, and the active ingredient may be appropriately used according to a conventional method. A mixing amount of the active ingredient may be suitably determined depending on its purpose of use (for prevention or treatment).

As an inhalant for parenteral administration, an aerosol, a powder for inhalation, or a liquid for inhalation is included, and such a liquid for inhalation may be dissolved or suspended in water or another suitable medium at the time of use. Such an inhalant is prepared in accordance with a known method. For example, a liquid for inhalation is formulated by suitably selecting a preservative (benzalkonium chloride, paraben, etc.), a coloring agent, a buffer (sodium phosphate, sodium acetate, etc.), an isotonic agent (sodium chloride, concentrated glycerin, etc.), a thickening agent (carboxyvinyl polymer, etc.), and an absorption enhancer as needed.

A powder for inhalation is formulated by suitably selecting a lubricant (stearic acid and a salt thereof, etc.), a binder (starch, dextrin, etc.), an excipient (lactose, cellulose, etc.), a coloring agent, a preservative (benzalkonium chloride, paraben, etc.), and an absorption promoter as needed.

The inhalant composition may be administered using an inhalant apparatus, the inhalant apparatus is an apparatus that can deliver the composition to a subject such as lung tissue thereof, for example, an inhaler, a nebulizer or a ventilator. To administer a liquid for inhalation, a common spray (atomizer or nebulizer) is used, and to administer a powder for inhalation, usually, an inhalation dispenser for powder medicine is used.

As another aspect of the present invention, the present invention provides a food composition for preventing or alleviating eosinophilic inflammatory diseases or Th2 hypersensitivity immune diseases, comprising Lactococcus lactis-derived vesicles as an active ingredient.

In the present invention, the food composition may be a health functional food composition, but the present invention is not limited thereto.

The vesicles derived from Lactococcus lactis according to the present invention may be used by adding the vesicles derived from Lactococcus lactis as is to food or may be used together with other foods or food ingredients, but may be appropriately used according to a typical method. The mixed amount of the active ingredient may be suitably determined depending on the purpose of use thereof (for prevention or alleviation). In general, when a food or beverage is prepared, the Lactococcus lactis-derived vesicles of the present invention is added in an amount of 15 wt % or less, preferably 10 wt % or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above-mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

As another aspect of the present invention, the present invention provides a quasi-drug composition for preventing or alleviating eosinophilic inflammatory diseases or Th2 hypersensitivity immune diseases, comprising Lactococcus lactis-derived vesicles as an active ingredient.

The term “quasi-drug” refers to items with a less action than pharmaceuticals among items used for the purpose of diagnosing, curing, alleviating, reducing, treating or preventing a human or animal disease, and for example, according to the Pharmaceutical Law, quasi-drugs are items excluding those used for the purpose of pharmaceuticals, and include products used to treat or prevent human or animal diseases, and products that have little or no direct action on a human body.

When the composition of the present invention is included in a quasi-drug for the purpose of preventing or alleviating an eosinophilic inflammatory disease, the composition may be comprised as it is or used together with other quasi-drug components, and may be appropriately used according to a conventional method. The mixing amount of the active ingredient may be suitably determined depending on the purpose of use.

The quasi-drug of the present invention may contain various types of bases and additives necessary for preparation depending on a dosage form, and the type and amount of each component may be easily selected by one of ordinary skill in the art.

The quasi-drug composition of the present invention may be prepared in the dosage form selected from the group consisting of, for example, a disinfection cleanser, a detergent, a detergent for kitchen, a detergent for cleaning, wet tissue, a detergent, soap, hand wash, a humidifier filler, a mask, an ointment, a filter filler, and a portable product that supplies air or oxygen temporarily by inhaling air or oxygen directly or indirectly, but the present invention is not limited thereto.

As another aspect of the present invention, the present invention provides a composition for delivering a drug for treating respiratory diseases, comprising Lactococcus lactis-derived vesicles as an active ingredient.

The “drug delivery” used herein refers to all means or behaviors which are delivered by loading a drug for treating respiratory diseases in L. lactis-derived vesicles according to the present invention to deliver a drug to a specific organ, tissue, cells, or cell organelle.

In the composition for delivering a drug for treating respiratory diseases according to the present invention, the respiratory diseases may comprise respiratory diseases exhibiting Th2 cell hypersensitivity and respiratory diseases characterized by excessive mucus secretion to the respiratory organs, and the type of composition is not limited.

In the present invention, there is no limit to the type of drug for treating respiratory diseases.

As another aspect of the present invention, the present invention provides a method of preventing or treating eosinophilic inflammatory diseases or Th2 hypersensitivity immune diseases, comprising administering a composition comprising Lactococcus lactis-derived vesicles as an active ingredient to a subject.

As another aspect of the present invention, the present invention provides a use of a composition comprising Lactococcus lactis-derived vesicles as an active ingredient for preventing or treating eosinophilic inflammatory diseases or Th2 hypersensitivity immune diseases.

As another aspect of the present invention, the present invention provides a use of Lactococcus lactis-derived vesicles for preparing a medicine for preventing or treating eosinophilic inflammatory diseases or Th2 hypersensitivity immune diseases.

As used herein, the “subject” refers to a subject in need of treatment of a disease, and more specifically, refers to a mammal such as a human or a non-human primate, a mouse, a rat, a dog, a cat, a horse, and a cow.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

In the present invention, when a part “comprises” a certain component, it means that, unless otherwise stated, it can further include other components, not excluding other components.

Hereinafter, preferred Examples and Experimental Examples for helping the understanding of the present invention will be suggested. However, the following Examples and Experimental Examples are provided only to more easily understand the present invention, and the contents of the present invention are not limited by the following Examples and Experimental Examples.

### EXAMPLES

### Example 1. Bacterial Culture and Isolation of Extracellular Vesicles

Lactococcus lactis and Bifidobacterium breve strains were cultured in self-prepared media until an optical density at 600 nm reached 1.5 under an anaerobic condition. To isolate extracellular vesicles (EVs), a bacterial culture medium was centrifuged at 10,000 g for 20 minutes, and the supernatant was filtered through a 0.45-μm vacuum filter. The filtrate was concentrated using QuixStand (GE Healthcare, U.K.), and filtered through a 0.22-μm bottle-top filter (Sigma-Aldrich, U.S.). Subsequently, the filtrate was pelleted by ultracentrifugation in a 45 Ti rotor (Beckman Coulter, U.S.) at 150,000 g for 2 hours at 4° C. The final pellet was resuspended in phosphate buffered saline and stored at −80° C., and a JEM1011 microscope (JEOL, Japan) was used to observe EV morphology. In addition, an EV size was measured using Zetasizer Nano S (Malvern Instruments, U.K.). An EV protein pattern was analyzed through sodium dodecyl sulfate-polyacrylamide gel electrophoresis.

### Example 2. Th2-Hypersensitivity Immune Disease Mouse Model

To induce Th2 hypersensitivity immune disease mouse models, female 6-week-old BALB/c mice (Jackson Laboratory, U.S.) were administered intraperitoneally with 75 μg of ovalbumin (OVA; Sigma-Aldrich) and 2 mg of aluminum hydroxide (alum; Thermo FisherScientific, U.S.) for sensitization. Subsequently, for a challenge, 50 μg of OVA was intranasally injected five times. During challenge, the mice were intraperitoneally administered with 10 μg of dexamethasone (Dexa; Sigma-Aldrich), or intranasally treated with 10 μg of EVs. To measure airway hypersensitivity to inhaled methacholine (Sigma-Aldrich), a flexiVent System (SCIREQ, Canada) was used, and to count the number of immune cells of BALF, Diff-quick staining (Dade Behring, Swiss) was performed. For histology, lung tissues stained with H&E or PAS were examined using ImageJ (National Institutes of Health, Bethesda, U.S.).

### Example 3. Isolation of Mouse Lune T Cells and Stimulation

Lung cells were isolated using immunomagnetic cell sorting (Miltenyi Biotec Inc, U.S.). These cells (5×10⁵) were inoculated into a 24-well plate (TPP, Swiss), and each cell was stimulated with mouse anti-CD3/CD28 antibody (1 μg/mL each; eBioscience, U.S.) for 24 hours.

### Example 4. Immune Cell Stimulation in Human Peripheral Blood

To isolate immune cells, venous blood of an asthmatic patient was collected in a vacuum tube containing acid citrate dextrose solution (BD Biosciences, U. S.). A layer of blood was added to a Lymphoprep solution (Axis-Shield, Norway), and centrifuged at 800×g for 25 minutes at 20° C. Then, the layer containing peripheral blood mononuclear cells was separated and red blood cells were moved by hypotonic lysis. Finally, each of dendritic cells and CD4+ T cells was isolated using immunomagnetic cell sorting (Miltenyi Biotec Inc, U.S.). The dendritic cells were treated with 100 ng/mL human recombinant IL-1I3 (R&D Systems, U.S.), 10-6 M Dexa (Sigma-130 Aldrich), or 1 μg/mL of EVs for 24 hours, the CD4+ T cells were stimulated with human anti-CD3/CD28 antibody (1 μg/mL each; Thermo FisherScientific) for 24 hours regardless of the presence of 10-6 M Dexa (Sigma-Aldrich) or 1 μg/mL of EVs.

### Example 5. Enzyme-Linked Immunosorbent Assay (ELISA)

Levels of various cytokines such as IFN-γ, IL-4, IL-5, IL-6, IL-10, IL-12p70 and IL-13 were measured in BALF or the cell culture supernatant using a kit (R&D Systems) according to a manufacturer's recommendations.

### Example 6. Statistical Analysis

All statistical analyses were performed using IBM SPSS software ver. 25.0 (IBM Corp., U.S.), and P<0.05 was considered statistically significant. GraphPad Prism 8.0 software (GraphPad Inc., U.S.) was used to plot a graph.

### EXPERIMENTAL EXAMPLES

### Experimental Example 1. Isolation and Characterization of Probiotic-Derived EVs

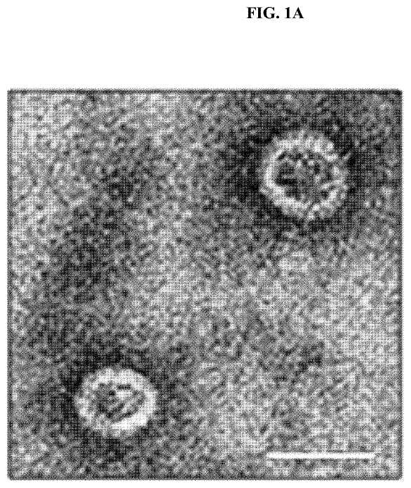

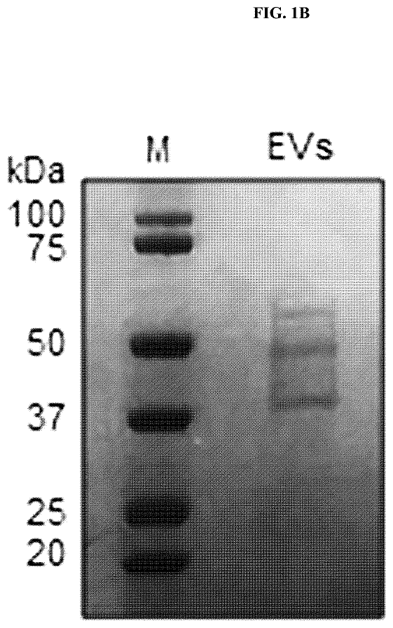

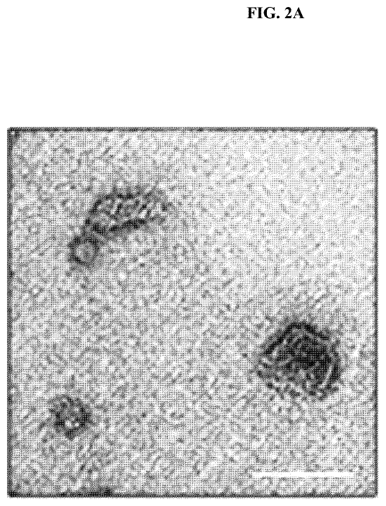

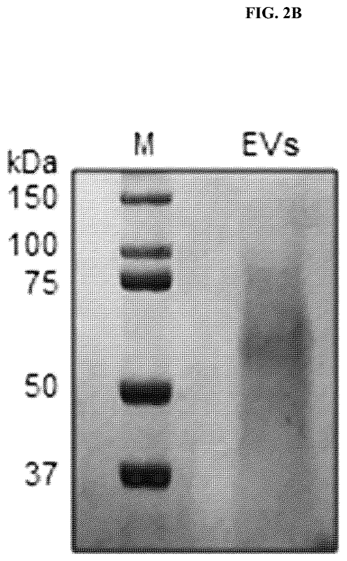

Lactococcus lactis-derived vesicles and Bifidobacterium breve-derived vesicles were purified in bacterial culture media, and to confirm whether EVs have a spherical lipid bilayer with an intact form, EVs were observed using a transmission electron microscope. As a result, as shown in FIGS. 1A and 2A, both L. lactis-derived vesicles and B. breve-derived vesicles showed a well-structured membrane formed in a bilayer.

In addition, when comparing protein patterns of EVs, as shown in FIG. 1B, L. lactis-derived vesicles showed several protein bands, but as shown in FIG. 2B, B. breve-derived vesicles showed a single band.

### Experimental Example 2. Therapeutic Effect of L. lactis-Derived Vesicles in Th2 Hypersensitivity Immune Disease Mouse Model

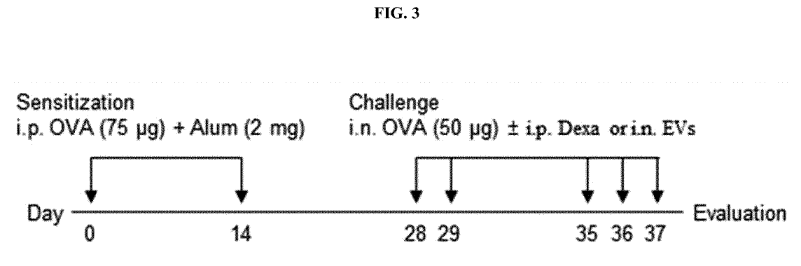

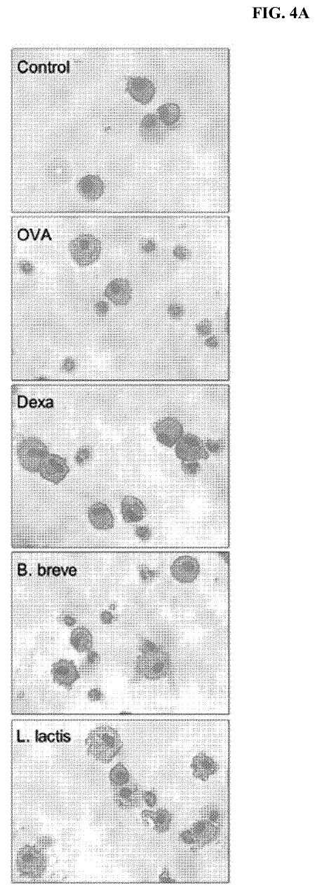

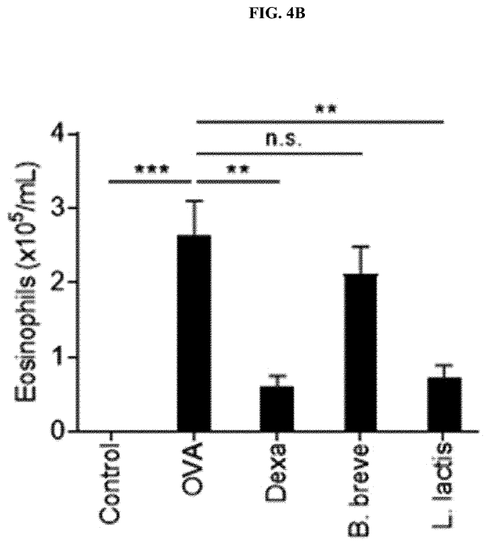

To investigate the role of EVs by comparing Dexa in Th2 hypersensitivity immune disease models, mice were treated with several medicines as shown in FIG. 3. During challenge, when L. lactis-derived vesicles, B. breve-derived vesicles, or Dexa was administered, as shown in FIGS. 4A and 4B, the number of eosinophils in BALF was significantly reduced by dexamethasone (Dexa) and L. lactis-derived vesicles, but not by B. breve-derived vesicles.

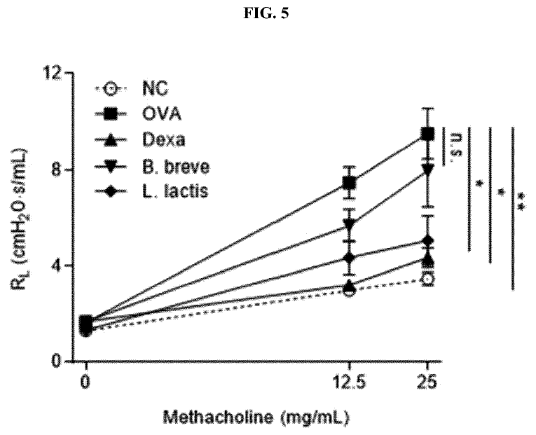

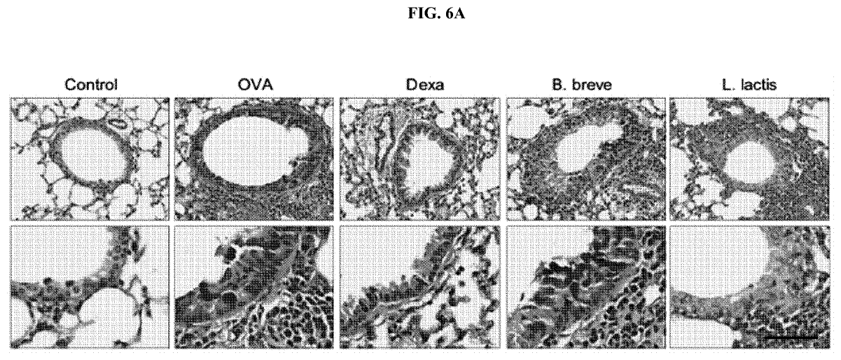

In addition, as shown in FIG. 5, a change in lung function (airway hypersensitivity) induced by Th2 hypersensitivity in the Th2 hypersensitivity immune disease model was significantly inhibited by Dexa and L. lactis-derived vesicles, but not by B. breve-derived vesicles.

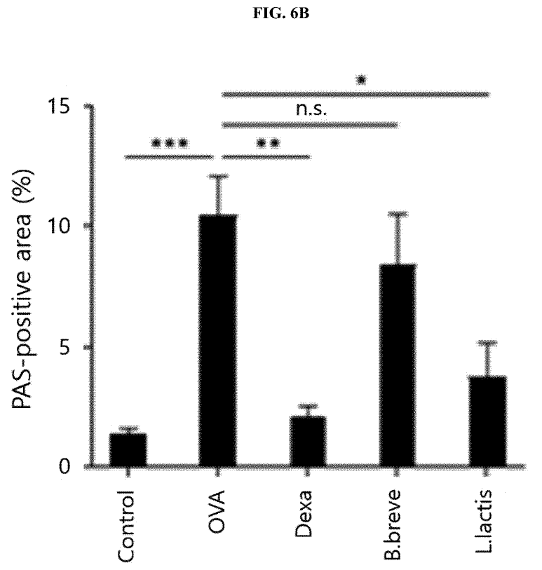

In addition, as shown in FIGS. 6A and 6B, a histological change such as mucus secretion induced by Th2 hypersensitivity in the Th2 hypersensitivity immune disease model was significantly inhibited by Dexa and L. lactis-derived vesicles, but not by B. breve-derived vesicles.

From the above results, it was seen that eosinophilic inflammation caused by immunological hypersensitivity and functional and histopathological changes caused thereby can be effectively treated using L. lactis-derived vesicles.

### Experimental Example 3. Immunomodulatory Effect of L. lactis-Derived Vesicles in Th2 Hypersensitivity Immune Disease Mouse Model

To evaluate the effects of probiotics-derived EVs and dexamethasone, which is representative corticosteroid as a control drug, on Th2 hypersensitivity, cytokines secreted from Th1 and Th2 cells in Th2 hypersensitivity immune disease mouse models were assessed.

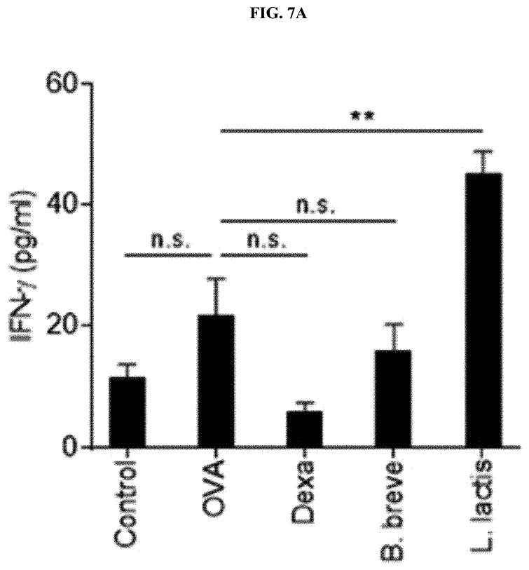

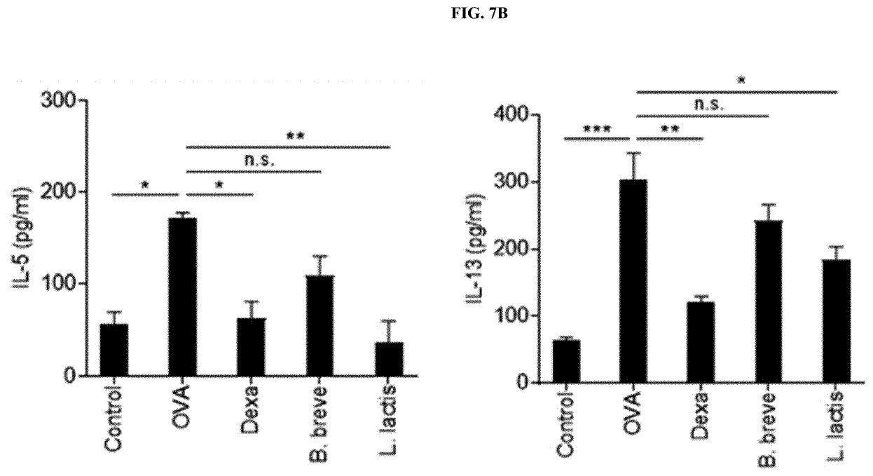

As a result, as shown in FIG. 7A, there was no significant difference in concentration of IFN-γ, which is a Th1 cytokine in the airway lavage fluid, by the administration of Dexa and B. breve-derived vesicles compared to the disease group, but the concentration was significantly increased by the administration of L. lactis-derived vesicles compared to the disease group.

On the other hand, as shown in FIG. 7B, there was no significant difference in concentrations of IL-5 and IL-13, which are Th2 cytokines in the airway lavage fluid, by the administration of B. breve-derived vesicles compared to the disease group, but the concentrations were significantly reduced by the administration of Dexa and L. lactis-derived vesicles compared to the disease group.

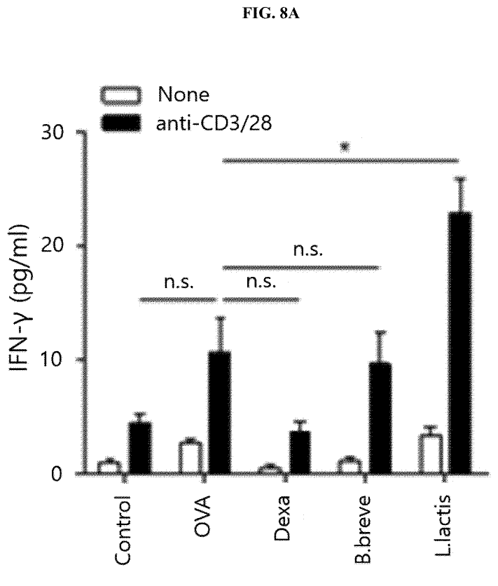

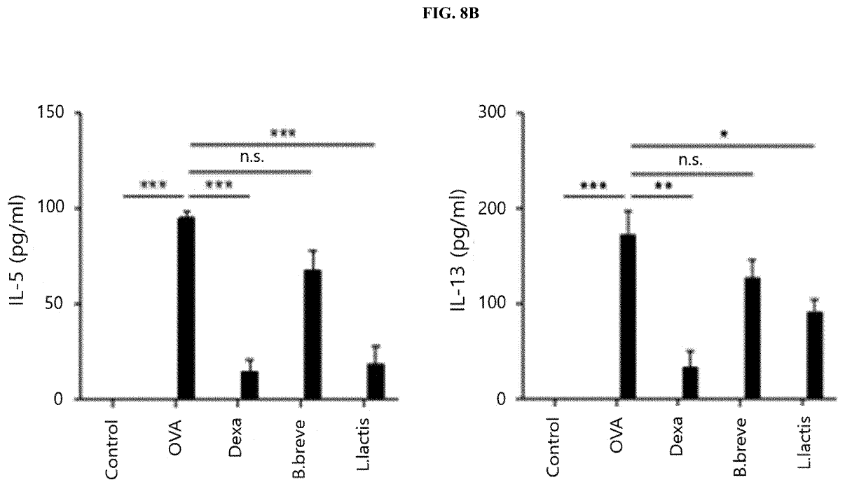

In addition, as a result of evaluating a cytokine secretion pattern after isolating T cells from lung tissue of a Th2 hypersensitivity immune disease mouse and stimulating the cells with anti-CD3/28, as shown in FIG. 8A, there was no significant difference in IFN-γ secretion in T cells by the administration of B. breve-derived vesicles compared to the disease group, but the IFN-γ secretion in T cells was inhibited by Dexa administration, and significantly increased by the administration of L. lactis-derived vesicles. On the other hand, as shown in FIG. 8B, there was no significant difference in secretion of Th2 cytokines, IL-5 and IL-13, in T cells by the administration of B. breve-derived vesicles compared to the disease group, but the secretion of IL-5 and IL-13 was significantly reduced by the administration of Dexa and L. lactis-derived vesicles.

From the above result, compared to Dexa inhibiting eosinophilic inflammation via a mechanism of inhibiting the overall immune function, it was able to be seen that L. lactis-derived vesicles inhibit eosinophilic inflammation by an action of increasing Th1 immune response but inhibiting Th2 immune response.

### Experimental Example 4. Mechanism of Inhibiting Th2 Hypersensitivity Response of L. lactis-Derived Vesicles

In the process of differentiating naive T cells into Th1 or Th2 cells, it has been well known that IL-12 secreted from dendritic cells, which are antigen-presenting cells, induces differentiation into Th1 cells, and IL-4 induces differentiation into Th2 cells. In this experimental example, to evaluate a mechanism of L. lactis-derived vesicles, which inhibits Th2 hypersensitivity response, an experiment was conducted by isolating T cells and dendritic cells from peripheral blood of normal persons.

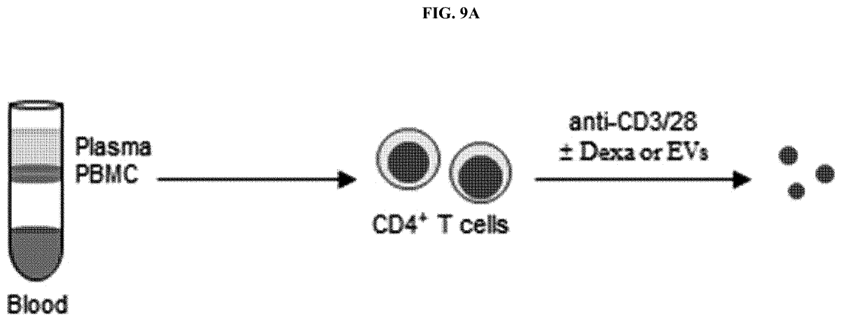

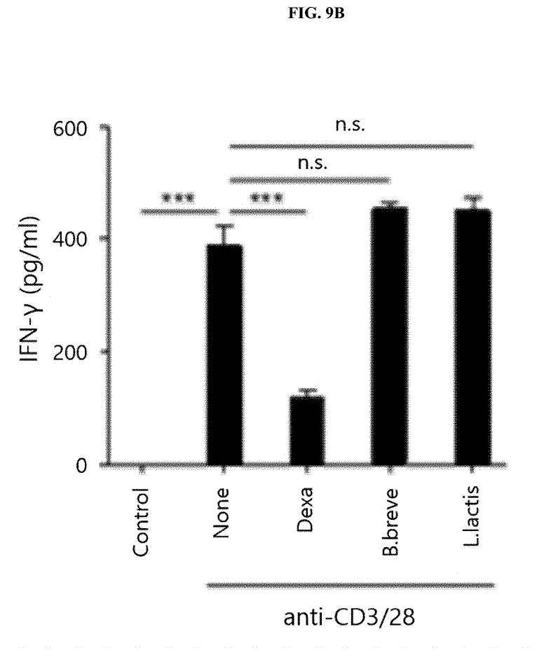

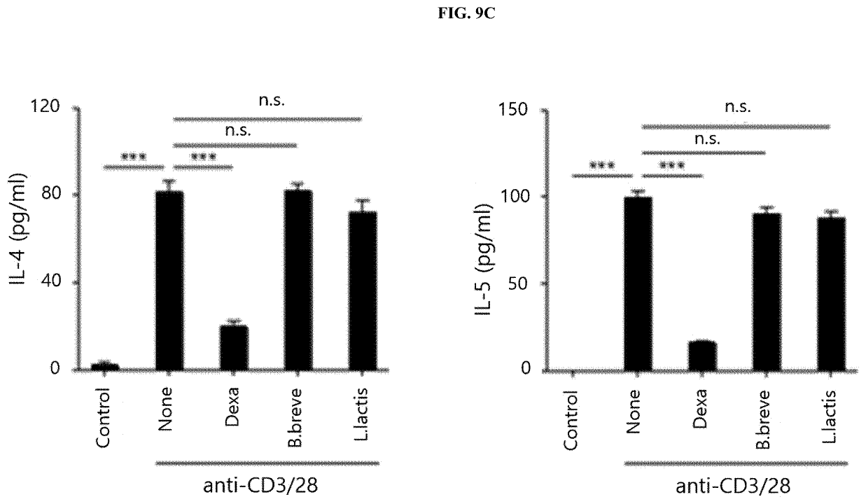

In one experimental example, as a result of evaluating a cytokine secretion pattern after anti-CD3/28 stimulation to T cells isolated from peripheral blood, shown in FIG. 9A, it was able to be seen that, as shown in FIG. 9B, Dexa inhibited IFN-γ secretion in T cells, but L. lactis-derived vesicles and B. breve-derived vesicles had no effect on IFN-γ secretion. In addition, as shown in FIG. 9C, the secretion of IL-4 and IL-5, which are Th2 cytokines, in T cells was inhibited by Dexa, but there were no changes by Lactococcus- and Bifidobacterium-derived vesicles.

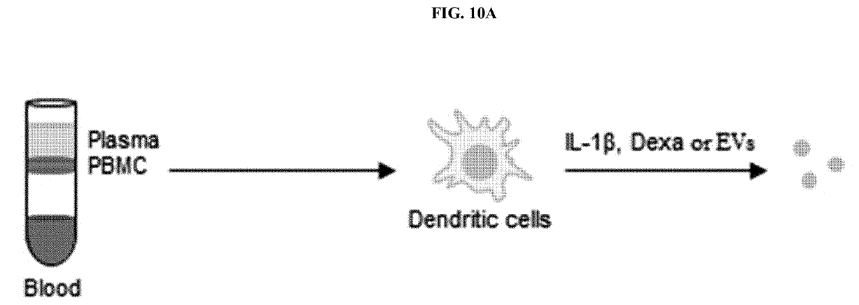

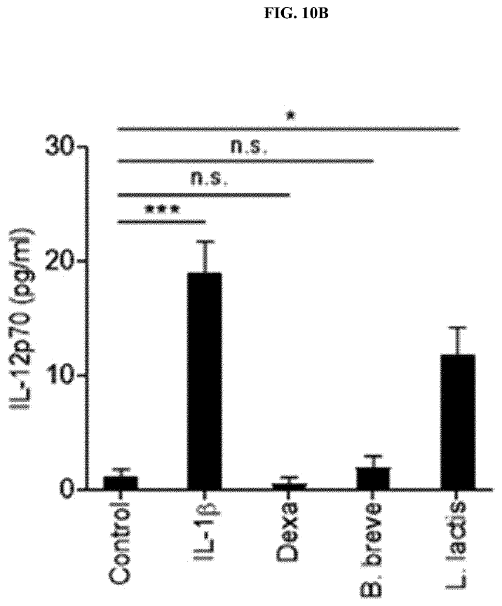

In another experimental example, as shown in FIG. 10A, an IL-12 secretion pattern inducing differentiation into Th1 cells in dendritic cells isolated from peripheral blood was evaluated. As a result, as shown in FIG. 10B, IL-12p70 secretion was not influenced by Dexa and Bifidobacterium-derived vesicles, but significantly increased by Lactococcus-derived vesicles and IL-1β, which is a control drug.

From the above result, it was able to be seen that L. lactis-derived vesicles act on dendritic cells, which are antigen-presenting cells, rather than directly acting on T cells, to induce IL-12 secretion inducing Th1 immune response, inhibiting Th2 hypersensitivity response.

The above-described description of the present invention is provided for illustrative purposes, and those of ordinary skill in the art to which the present invention pertains will understand that the present invention can be easily modified into other specific forms without changing the technical spirit or essential features of the present invention. Therefore, it should be understood that the above-described Examples are illustrative only in all aspects and are not restrictive.

### INDUSTRIAL APPLICABILITY

When administered to a Th2 hypersensitivity immune disease mouse model, L. lactis-derived vesicles according to the present invention inhibit eosinophilic inflammation, and functional and histopathological changes caused thereby, Th1 immune response is induced, and Th2 immune response is inhibited, so the L. lactis-derived vesicles are expected to be effectively used in a composition for preventing, treating or alleviating eosinophilic inflammatory diseases and Th2 hypersensitivity immune diseases.

1.-34. (canceled)

35. A method for treating or alleviating eosinophilic inflammatory disease or Th2 hypersensitivity immune disease, comprising administering to a subject in need thereof a composition comprising Lactococcus lactis-derived vesicles as an active ingredient.

36. The method of claim 35, wherein the eosinophilic inflammatory disease is allergic inflammatory disease comprising eosinophilic drug allergy, eosinophilic asthma, allergic rhinitis, or atopic dermatitis.

37. The method of claim 35, wherein the eosinophilic inflammatory disease is eosinophilic inflammatory disease of unknown comprising eosinophilic cardiomyopathy, eosinophilic colitis, eosinophilic enteritis, eosinophilic esophagitis, eosinophilic gastritis, eosinophilic pneumonia, eosinophilic bronchitis, eosinophilic fasciitis, hypereosinophilic syndrome, or Churg-Strauss syndrome or eosinophilic granulomatosis with polyangiitis.

38. The method of claim 35, wherein the Th2 hypersensitivity immune disease comprises atopic dermatitis, allergic conjunctivitis, allergic rhinitis, allergic asthma, hypersensitivity pneumonitis, food allergy, drug allergy, or anaphylaxis.

39. The method of claim 35, wherein the eosinophilic inflammatory disease is respiratory disease showing type 2 helper T (Th2) cell hypersensitivity.

40. The method of claim 39, wherein the respiratory disease showing Th2 cell hypersensitivity comprises atopic asthma, allergic rhinitis, eosinophilic bronchitis, or hypersensitivity pneumonitis.

41. The method of claim 35, wherein the eosinophilic inflammatory disease or Th2 hypersensitivity immune disease is respiratory disease characterized by excessive secretion of mucus to the respiratory organs.

42. The method of claim 41, wherein the respiratory disease characterized by excessive secretion of mucus to the respiratory organs comprises chronic rhinitis, chronic sinusitis, or chronic bronchitis.

43. The method of claim 35, wherein the Lactococcus lactis-derived vesicles inhibit airway hyperresponsiveness.

44. The method of claim 35, wherein the vesicles have an average diameter of 10 to 200 nm.

45. The method of claim 35, wherein the vesicles are secreted naturally or artificially from Lactococcus lactis.

46. The method of claim 35, wherein the composition is a pharmaceutical composition, an inhalant composition, a food composition, or a quasi-drug composition.

47. A method of delivering a drug for treating respiratory disease, the method comprising administering a composition comprising Lactococcus lactis-derived vesicles containing a drug for treating respiratory diseases to be targeted as an active ingredient to a subject in need thereof.
